(19) United States
(12) Patent Application Publication
(10) Pub. No.: US 2025/0369893 A1
(43) Pub. Date: Dec. 4, 2025

(54) COLORIMETRIC SENSOR FILM AND MODULAR SENSOR DEVICE USING THE SAME

(71) Applicant: CALYX, INC., Berkeley, CA (US)

(72) Inventors: Benson FAN, Taipei City (TW); Po-Jui CHIU, Taipei City (TW); Ming-Yuan TSAI, Taipei City (TW); I-Ting CHEN, Taipei City (TW)

(73) Assignee: CALYX, INC., Berkeley, CA (US)

(21) Appl. No.: 18/732,273

(22) Filed: Jun. 3, 2024

(51) Int. Cl. G01N 21/78; G01N 33/00

(52) U.S. Cl. CPC G01N 21/78; G01N 33/0027

(57) ABSTRACT

A colorimetric sensor film is disclosed. The colorimetric sensor film comprises a substrate and a sensing layer formed on the substrate, of which the sensing layer is able to change a visual color thereof after adsorbing a specific chemical substance contained in a target gas through physisorption or chemisorption. Moreover, a modular sensor device is also disclosed. The modular sensor device comprises a component housing containing a detection chamber and a reference chamber, a light source, said colorimetric sensor film, a detection optical sensor, a reference sensor film, a reference optical sensor, and a processor, in which the reference sensor film is able to exhibit a very low visual color change under different temperatures and humidity conditions. Therefore, the modular sensor device can detect at least one kind of gas and/or suspended matter in air with high selectivity, sensitivity and accuracy.

### BACKGROUND OF THE INVENTION

### 1. Field of the Invention

The present invention relates to the technology field of colorimetric sensors, and more particularly to a colorimetric sensor film and a modular sensor device using the colorimetric sensor film.

### 2. Description of the Prior Art

It is known that gas sensors are currently used to dynamically monitor environmental pollution, which is a common concern in everyday life, industry, academia and research circles. Nowadays, gas sensors are typically classified into various types based on the type of the sensing element it is built with, including: (1) MOS-based gas sensor, (2) optical gas sensor, (3) electrochemical gas sensor, (4) capacitance-based gas sensor, (5) colorimetric gas sensor, and (5) acoustic based gas sensor.

Among the various gas sensors available for environmental pollution monitoring, colorimetric gas sensors remain advantageous in that the human eye can be used for signal transduction, rather than extensive instrumentation. Though colorimetric gas sensors currently exist for a range of analytes, most are based upon employing dyes or colored chemical indicators for detection. Such compounds are typically selective, meaning arrays are necessary to enable detection of various classes of compounds. Moreover, many of these systems have lifetime limitation issues, due to photo-bleaching or undesirable side reactions.

According to above descriptions, it is realized that there is still room for improvement in the conventional colorimetric gas sensors. Accordingly, inventors of the present application have made great efforts to make inventive research and eventually provided a colorimetric sensor film and a modular sensor device using the colorimetric sensor film.

### SUMMARY OF THE INVENTION

The first objective of the present invention is to disclose a colorimetric sensor film capable of changing a visual color after adsorbing a specific chemical substance contained in a target gas through physisorption or chemisorption.

The second objective of the present invention is to disclose a modular sensor device using the foregoing colorimetric sensor film and a reference sensor film, wherein the reference sensor film is able to exhibit a very low visual color change under different temperatures and humidity conditions. Therefore, the modular sensor device can detect at least one kind of gas and/or suspended matter in air with high selectivity, sensitivity and accuracy.

For achieving the objectives mentioned above, the present invention provides an embodiment of the colorimetric sensor film, comprising:
a substrate; and
a sensing layer, being formed on the substrate, and being able to change a visual color thereof after adsorbing a specific chemical substance contained in a target gas through physisorption or chemisorption;
wherein the sensing layer is made from a material that comprises at least one selected from a group consisting of carbon quantum dots, single-walled carbon nanotubes (SWCNTs), multi-walled carbon nanotubes (MWCNTs), graphene quantum dots (GQDs), M13 bacteriophage, short chain peptide, porphyrin, porphyrin derivative, metal-organic framework (MOF), phthalocyanine, phthalocyanine derivative, phthalein, and pH dye.

In one practicable embodiment, a protective coating is formed on the substrate for cladding the sensing layer.

In another one practicable embodiment, the sensing layer is enclosed in an encapsulation layer that is formed on the substrate.

In one embodiment, the substrate is selected from a group consisting of silicon substrate, glass substrate, porous substrate, polymer substrate, and silicon wafer, of which said porous substrate is selected from a group consisting of Al₂O₃ substrate, MOFs substrate, TiO₂ substrate, and SiO₂ substrate, and said polymer substrate is selected from a group consisting of polytetrafluoroethylene (PTFE) substrate, polyvinylidene fluoride (PVDF) substrate, and polyethylene substrate.

In one embodiment, the substrate has a surface selected from a group consisting of smooth surface and rough surface, and the surface has a reflectivity of at least 80% in a wavelength range between 400-700 nm.

In further one practicable embodiment, an additional layer, made of at least one material selected from a group consisting of gold (Au), palladium (Pd), platinum (Pt), chromium (Cr), titanium (Ti), and aluminum (Al), is deposited on the substrate and has a thickness in a range between 100 nm and 300 nm.

In one embodiment, an adhesion layer comprising chromium (Cr) and titanium (Ti) is formed between the substrate and the additional layer, and the adhesion layer has a thickness in a range between 5 nm and 30 nm.

For achieving the objectives mentioned above, the present invention also provides an embodiment of the modular sensor device, comprising:
a component housing, being provided with a detection chamber and a reference chamber isolated from the detection chamber therein, and being further provided with a first through-hole in communication with the detection chamber and a second through-hole in communication with the reference chamber thereon, such that a target gas is able to flow into the detection chamber and the reference chamber via the first through-hole and the second through-hole;
a light source, being disposed in the component housing, and being configured for emitting an incident light that enters the detection chamber and the reference chamber;
a colorimetric sensor film, being disposed in the detection chamber so as to be exposed to the target gas, and comprising a substrate and a sensing layer formed on the substrate; wherein the sensing layer is able to change a visual color thereof after adsorbing a specific chemical substance contained in the target gas through physisorption or chemisorption, such that after the incident light is directed to the sensing layer in the detection chamber, a reflective light response to the visual color is generated, thereby being adopted as a detection light;
a detection optical sensor, being disposed in the component housing so as to face the colorimetric sensor film, and being configured to generate a detected spectral signal after receiving the detection light;
a reference sensor film, being disposed in the reference chamber so as to be exposed to the target gas; wherein after the incident light is directed to the reference sensor film, a reflective light of the incident light is generated from a light incident surface of the reference sensor film, thereby being adopted as a reference light;
a reference optical sensor, being disposed in the component housing so as to face the reference sensor film, and being configured to generate a reference spectral signal after receiving the reference light; and
a processor, being coupled to the detection optical sensor and the reference optical sensor, and being configured to:
receive the detected spectral signal so as to correspondingly generate a detection data;
receive the reference spectral signal so as to correspondingly generate a reference data; and
generate a sensing data by comparing the detection data with the reference data;
wherein the sensing layer is made from a material that comprises at least one selected from a group consisting of carbon quantum dots, single-walled carbon nanotubes (SWCNTs), multi-walled carbon nanotubes (MWCNTs), graphene quantum dots (GQDs), M13 bacteriophage, short chain peptide, porphyrin, porphyrin derivative, metal-organic framework (MOF), phthalocyanine, phthalocyanine derivative, phthalein, and pH dye.

In one embodiment, the substrate has a surface selected from a group consisting of smooth surface and rough surface, and the surface has a reflectivity of at least 80% in a wavelength range of 400-700 nm, and the substrate is selected from a group consisting of silicon substrate, glass substrate, porous substrate, polymer substrate, and silicon wafer.

In one practicable embodiment, a protective coating is formed on the substrate for cladding the sensing layer.

In another one practicable embodiment, the sensing layer is enclosed in an encapsulation layer that is formed on the substrate.

In one embodiment, a first recess across the first through-hole is formed on the component housing, and a second recess across the second through-hole is formed on the component housing, such that the colorimetric sensor film and the reference sensor film are positioned in the first recess and the second recess, respectively.

In one embodiment, a first optical path between the light source and the colorimetric sensor film has a first length, and a second optical path between the light source and the reference sensor film has a second length that is equal to the first length.

In further one practicable embodiment, the modular sensor device further comprises a cap member, comprising:
a third recess, being formed on a bottom of the cap member, and having a first bottom through-hole;
a fourth recess, being formed on the bottom of the cap member, and having a second bottom through-hole; and
a fifth recess, being formed on the bottom of the cap member, and having a third bottom through-hole;
wherein the cap member is disposed in the component housing for accommodating the detection optical sensor and the reference optical sensor and facing the colorimetric sensor film and the reference sensor film by the bottom thereof;
wherein the light source is disposed to face the third bottom through-hole, and the detection optical sensor and the reference optical sensor being disposed to face the first bottom through-hole and the second bottom through-hole, respectively.

In further one practicable embodiment, the modular sensor device further comprises a circuit board connected to an opening of the cap member, wherein the detection optical sensor, the reference optical sensor, and the processor are disposed on the circuit board.

In further one practicable embodiment, the modular sensor device further comprises a temperature sensor and a humidity sensor disposed in the detection chamber or the reference chamber, and the temperature sensor and the humidity sensor are coupled to the processor.

In further one practicable embodiment, the modular sensor device further comprises:
a diffusion lens disposed in the fifth recess;
a first protective glass disposed in the third recess; and
a second protective glass disposed in the fourth recess.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a first side cross-sectional diagram of a colorimetric sensor film according to the present invention;

FIG. 2 shows a second side cross-sectional diagram of the colorimetric sensor film according to the present invention;

FIG. 3 shows a third side cross-sectional diagram of the colorimetric sensor film according to the present invention;

FIG. 4 shows a stereo diagram of a modular sensor device according to the present invention;

FIG. 5 shows a top view of the modular sensor device according to the present invention;

FIG. 6 shows an exploded view of the modular sensor device according to the present invention;

FIG. 7A shows a side cross-sectional view taken along section line A-A′ shown in FIG. 5; and

FIG. 7B shows a side cross-sectional view taken along section line B-B′ shown in FIG. 5.

### DETAILED DESCRIPTION OF THE PREFERRED EMBODIMENTS

To more clearly describe a colorimetric sensor film and a modular sensor device, embodiments of the present invention will be described in detail with reference to the attached drawings hereinafter.

### Colorimetric Sensor Film

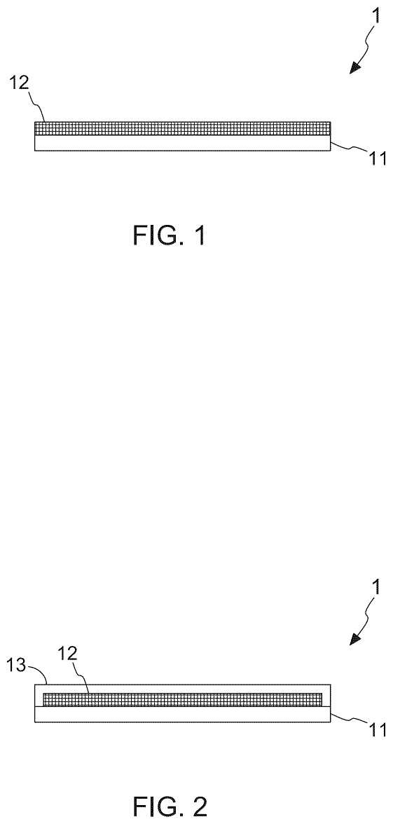

Selective gas sensors have been widely used to detect leakage of hazardous gases in living environments and industrial complexes. One example is the colorimetric gas sensor, in which colorimetric sensor element on a solid substrate react with specific gas species and exhibit color changes that can be used as a sensing signal. Colorimetric gas sensors are usually very cheap and enable one to selectively detect a broad range of different gas species. Accordingly, the present invention provides a colorimetric sensor film, and FIG. 1 illustrates a first side cross-sectional diagram of the colorimetric sensor film according to the present invention.

As FIG. 1 shows, the colorimetric sensor film 1 comprises a substrate 11 and a sensing layer 12 formed on the substrate 11, of which the sensing layer 12 has particular property of being able to change a visual color thereof after adsorbing a specific chemical substance contained in a target gas (e.g., air) through physisorption or chemisorption, and this color change is easily distinguishable and can be correlated to the concentration of the specific chemical substance in the target gas. In the case of physisorption, the interaction between the sensing layer 12 and the specific chemical substance is slightly exothermic, leading to a favorable equilibrium where the specific chemical substance is adsorbed onto the sensing layer 12 without requiring any additional energy input. In one embodiment, the sensing layer 12 is made from a material, and the material can be, but is not limited to, carbon quantum dots, single-walled carbon nanotubes (SWCNTs), multi-walled carbon nanotubes (MWCNTs), graphene quantum dots (GQDs), M13 bacteriophage, short chain peptide, porphyrin, porphyrin derivative, metal-organic framework (MOF), phthalocyanine, phthalocyanine derivative, phthalein, pH dye, or a combination of aforesaid two or more materials.

For instance, porphyrin is aromatic macrocycle constituted by four pyrrole rings linked by methine bridges. The four inner core nitrogen atoms represent probably the most versatile ligand system, able to coordinate almost all the elements of the Periodic Table. The peripheral positions of the macrocycle can be further decorated by additional peripheral substituents. The interplay between the aromatic ring, the metal ion, and the peripheral groups gives rise to specific and unique functionalities that can be exploited in a number of different applications. From this point of view, a porphyrin thin film can be modified to target different gas chemical compounds through organic or wet chemistry techniques. By selectively modifying the composition or structure of the film, it becomes tailored to interact specifically with the desired chemical compound. This modification process can involve organic functional groups or chemical agents that enhance the sensitivity and selectivity of the film towards the target compound.

On the other hand, phthalocyanine is an organic compound having a structure similar to the porphyrin structure, except that all four pyrrole subunits are fused to an additional benzene ring. Phthalocyanines have been regarded as the most important class of colorimetric gas sensors because of their intense coloration power and strong binding ability with VOCs. As described in more detail below, the metallic porphyrins or phthalocyanines may be complexes containing a metal such as cobalt, iron, chromium, zinc, or magnesium, and may have a metal core coordinatively linked with neighboring porphyrins through oxygen-containing species (water molecules), leading to long-range ordered, one-dimensional nanorods, in which the porphyrin planes are perpendicular to the substrate plane, and the porphyrin nanorods are lying parallel to the surface.

In addition, Graphene quantum dots (GQDs), as recently emerging carbon-based materials, are graphene sheets smaller than 100 nm and are proposed to be promising substitutes for the heavy metal-containing semiconductor-based QDs. The GQDs have attracted more and more attention due to their special advantages, such as low toxicity, better surface grafting properties because of their T-x conjugated networks or surface groups, high fluorescence activity, robust chemical inertness, and excellent photostability. Recently, sensor film made of N-doped GQDs (NGQDs) is synthesized so as to be used in sensing VOCs. As described in more detail below, PEDOT:PSS/graphene quantum dots is made to be a sensitive, selective, facile, and low cost VOCs sensor film.

In one embodiment, the substrate 11 can be, but is not limited to, a silicon substrate, a glass substrate, a porous substrate, a polymer substrate, or a silicon wafer, and has a smooth surface (e.g., a polished surface) or a rough surface (e.g., an etch-treated surface). According to the present invention, the surface has a reflectivity of at least 80% in certain wavelengths between 400-700 nm. As described in more detail below, aforesaid polymer substrate such as, but not limited to, polytetrafluoroethylene (PTFE) substrate, polyvinylidene fluoride (PVDF) substrate, or polyethylene substrate. On the other hand, Al₂O₃ substrate, metallic organic framework (MOF) substrate, TiO₂ substrate, and SiO₂ substrate are examples of aforesaid porous substrate.

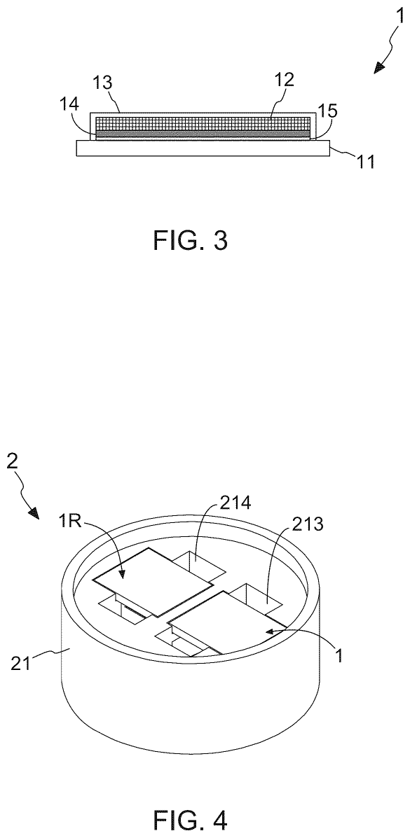

With continued reference to FIG. 2, there is shown a second side cross-sectional diagram of the colorimetric sensor film according to the present invention. As FIG. 2 shows, when producing the colorimetric sensor film 1, it is suggested that either making the sensing layer 12 be enclosed in an encapsulation layer 13 formed on the substrate 11 or forming a protective coating on the substrate 11 to clad the sensing layer 12. In addition, FIG. 3 illustrates a third side cross-sectional diagram of the colorimetric sensor film according to the present invention. As FIG. 3 shows, in one practicable embodiment an additional layer 14 having a thickness of 100-300 nm is deposited on the substrate 11, and there is further an adhesion layer 15 with a thickness of 5-30 nm formed between the substrate 11 and the additional layer 14, wherein the adhesion layer 15 comprising chromium (Cr) and titanium (Ti) (i.e., Cr/Ti compound) and has a thickness in a range between 5 nm and 30 nm. On the other hand, the additional layer 14 is made of a material, and the material can be, but is not limited to gold (Au), palladium (Pd), platinum (Pt), chromium (Cr), titanium (Ti), aluminum (Al), or a combination of aforesaid two or more materials. For example, in case of using Au, Pd, Pt, or a compound of the aforesaid two or more material in the manufacture of said additional layer 14, Cr, Ti or Cr/Ti compound is adopted for forming said adhesion layer 15 between the substrate 11 and the additional layer 14. As such, after a test light emitted from a lighting device is directed to the sensing layer 12 and further passes through the sensing layer 12, the test light would be reflected by the additional layer 14.

### Modular Sensor Device

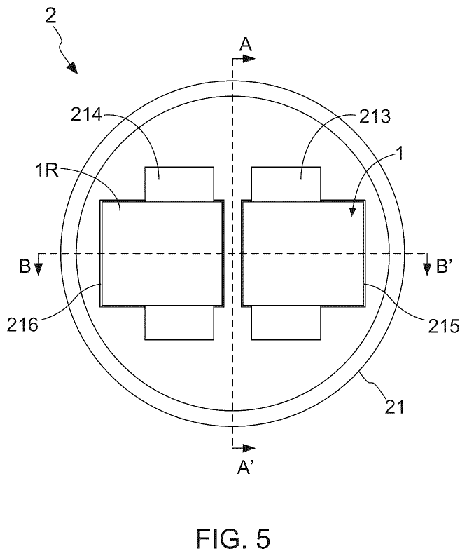

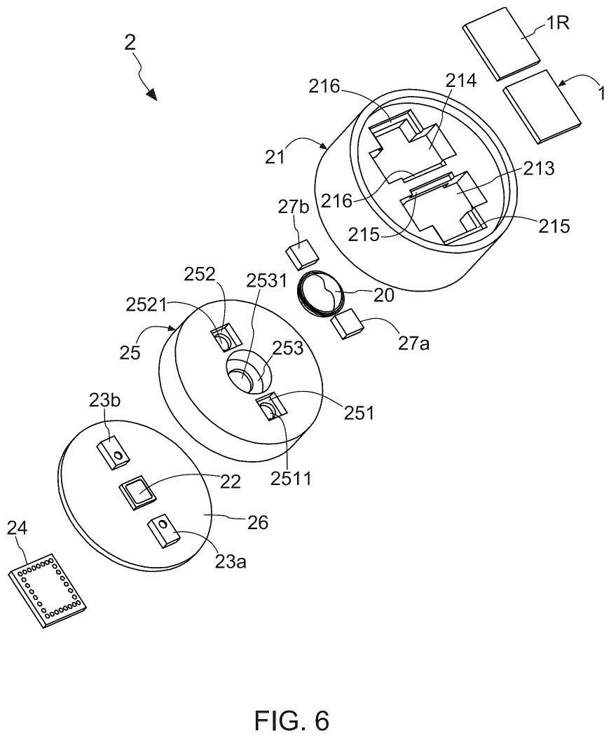

Subsequently, a modular sensor device using the foregoing colorimetric sensor film and a reference sensor film will be introduced and discussed below. FIG. 4 and FIG. 5 illustrate a stereo diagram and a top view of the modular sensor device according to the present invention. Moreover, FIG. 6 shows an exploded view of the modular sensor device. According to the present invention, the modular sensor device 2 principally comprises: a component housing 21, a light source includes a light source 22, one aforesaid colorimetric sensor film 1, a detection optical sensor 23a, a reference sensor film 1R, a reference optical sensor 23b, a processor 24, a cap member 25, and a circuit board 26.

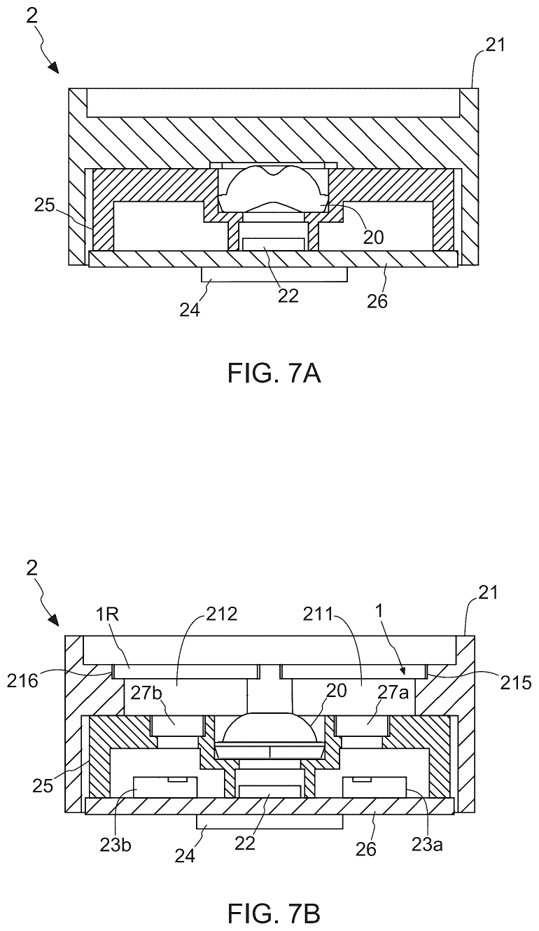

FIG. 7A provides a side cross-sectional view taken along section line A-A′ shown in FIG. 5, and FIG. 7B depicts a side cross-sectional view taken along section line B-B′ shown in FIG. 5. According to the present invention, the component housing 21 is provided with a detection chamber 211 and a reference chamber 212 isolated from the detection chamber 212 therein, and is further provided with a first through-hole 213 in communication with the detection chamber 211 and a second through-hole 214 in communication with the reference chamber 212 thereon, such that a target gas (e.g., air) is able to flow into the detection chamber 211 and the reference chamber 212 via the first through-hole 213 and the second through-hole 214. It is seen that a first recess 215 across the first through-hole 213 is formed on the component housing 21, and a second recess 216 across the second through-hole 214 is formed on the component housing 21, such that the colorimetric sensor film 1 and the reference sensor film 1R are positioned in the first recess 215 and the second recess 216, respectively. By such arrangements, the colorimetric sensor film 1 is disposed in the detection chamber 211 so as to be exposed to the target gas, and the reference sensor film 1R is disposed in the reference chamber 212 thereby being exposed to the target gas.

As FIG. 6, FIG. 7A, and FIG. 7B show, the cap member 25 is provided with a third recess 251, a fourth recess 252, and a fifth recess 253 on a bottom thereof, in which the third recess 251 has a first bottom through-hole 2511, the fourth recess 252 has a second bottom through-hole 2521, and the fifth recess 253 has a third bottom through-hole 2531. Particularly, the cap member 25 is disposed in the component housing 21 for accommodating the detection optical sensor 23a and the reference optical sensor 23b and facing the colorimetric sensor film 1 and the reference sensor film 1R by the bottom thereof. According to the present invention, the light source 22 is disposed to face the third bottom through-hole 2531, and the detection optical sensor 23a and the reference optical sensor 23b being disposed to face the first bottom through-hole 2511 and the second bottom through-hole 2521, respectively.

As such, the light source 22 is disposed in the component housing 21 and the cap member 25, and is controlled by the processor 24 so as to emit an incident light, such that the incident light is directed to enters the detection chamber 211 and the reference chamber 212 after being diffused by the diffusion lens 20 disposed in the fifth recess 253. Since the sensing layer 12 of the colorimetric sensor film 1 is able to exhibit a color change after adsorbing a specific chemical substance contained in the target gas through physisorption or chemisorption, such that after the first incident light is directed to the sensing layer 11 in the detection chamber 211, a reflective light response to the visual color is generated, thereby being adopted as a detection light. Moreover, the detection optical sensor 23a is disposed in the component housing 21 so as to face the colorimetric sensor film 1, and is configured to generate a detected spectral signal after receiving the detection light.

According to the present invention, the reference sensor film 1R can exhibit a very low visual color change under different temperatures and humidity conditions. Therefore, after the second incident light is directed to the reference sensor film 1R, a reflective light of the second incident light is generated from a light incident surface of the reference sensor film 1R, thereby being adopted as a reference light. Moreover, the reference optical sensor 23b is disposed in the component housing 21 so as to face the reference sensor film 1R, and is configured to generate a reference spectral signal after receiving the reference light.

It is worth noting that, FIG. 7A and FIG. 7B depict that a first optical path between the first lighting element 22a and the colorimetric sensor film 1 has a first length, and a second optical path between the second lighting element 22b and the reference sensor film 1R has a second length that is equal to the first length. As such, the processor 24 is able to receive the detected spectral signal and the reference spectral signal from the detection optical sensor 23a and the reference optical sensor 23b, respectively. After that, the processor 24 correspondingly generates a detection data and a reference data, and then generates a sensing data by comparing the detection data with the reference data. As described in more detail below, the circuit board 26 is connected to an opening of the cap member 25, and the detection optical sensor 23a, the reference optical sensor 23b, and the processor 24 are disposed on the circuit board 26. Moreover, a temperature sensor and a humidity sensor disposed in the detection chamber 211 or the reference chamber 212, and the temperature sensor and the humidity sensor being coupled to the processor 24. In addition, the modular sensor device 2 further comprises a first protective glass 27a and a second protective glass 27b, in which the first protective glass 27a is disposed in the third recess 251, and the second protective glass 27b is disposed in the fourth recess 252.

Therefore, through above descriptions, all embodiments and their constituting elements of the colorimetric sensor film 1 and the modular sensor device 2 using the colorimetric sensor film 1 proposed by the present invention have been introduced completely and clearly. In summary, the present invention includes the advantages of:
(1) the modular sensor device 2 is particularly designed to comprise a component housing 21 containing a detection chamber 211 and a reference chamber 212, a light source, a colorimetric sensor film 1 of the present invention, a detection optical sensor 23a, a reference sensor film 1R, a reference optical sensor 23b, and a processor 24, in which the colorimetric sensor film 1 is able to change a visual color thereof after adsorbing a specific chemical substance contained in a target gas, and the reference sensor film 1R may exhibit a very low visual color change under different temperatures and humidity conditions. Therefore, by utilizing a reference light collected from the reference sensor film 1R to calibrate a detection light 5 collected from the colorimetric sensor film 1, the modular sensor device 1 has a significant potential to detect at least one kind of gas and/or suspended matter in air with high selectivity, sensitivity and accuracy.
(2) There is no need for the modular sensor device 2 to include a large-volume gas detection chamber for multiple reflections, such that the modular sensor device 2 is allowed to be miniaturized.

Moreover, the above description is made on embodiments of the present invention. However, the embodiments are not intended to limit the scope of the present invention, and all equivalent implementations or alterations within the spirit of the present invention still fall within the scope of the present invention.

What is claimed is:

1. A colorimetric sensor film, comprising:
a substrate; and
a sensing layer, being formed on the substrate, and being able to change a visual color thereof after adsorbing a specific chemical substance contained in a target gas through physisorption or chemisorption;
wherein the sensing layer is made from a material that comprises at least one selected from a group consisting of carbon quantum dots, single-walled carbon nanotubes (SWCNTs), multi-walled carbon nanotubes (MWCNTs), graphene quantum dots (GQDs), M13 bacteriophage, short chain peptide, porphyrin, porphyrin derivative, metal-organic framework (MOF), phthalocyanine, phthalocyanine derivative, phthalein, and pH dye.

2. The colorimetric sensor film of claim 1, wherein a protective coating is formed on the substrate for cladding the sensing layer.

3. The colorimetric sensor film of claim 1, wherein the sensing layer is enclosed in an encapsulation layer that is formed on the substrate.

4. The colorimetric sensor film of claim 1, wherein the substrate is selected from a group consisting of silicon substrate, glass substrate, porous substrate, polymer substrate, and silicon wafer.

5. The colorimetric sensor film of claim 1, wherein the substrate has a surface selected from a group consisting of smooth surface and rough surface, and the surface has a reflectivity of at least 80% in a wavelength range of 400-700 nm.

6. The colorimetric sensor film of claim 1, wherein an additional layer is deposited on the substrate, and the additional layer is made of at least one material selected from a group consisting of gold (Au), palladium (Pd), platinum (Pt), chromium (Cr), titanium (Ti), and aluminum (Al).

7. The colorimetric sensor film of claim 6, wherein the additional layer has a thickness in a range between 100 nm and 300 nm.

8. The colorimetric sensor film of claim 6, wherein an adhesion layer comprising chromium (Cr) and titanium (Ti) is formed between the substrate and the additional layer, and the adhesion layer has a thickness in a range between 5 nm and 30 nm.

9. The colorimetric sensor film of claim 4, wherein said porous substrate is selected from a group consisting of Al₂O₃ substrate, MOFs substrate, TiO₂ substrate, and SiO₂ substrate.

10. The colorimetric sensor film of claim 4, wherein said polymer substrate is selected from a group consisting of polytetrafluoroethylene (PTFE) substrate, polyvinylidene fluoride (PVDF) substrate, and polyethylene substrate.

11. A modular sensor device, comprising:
a component housing, being provided with a detection chamber and a reference chamber isolated from the detection chamber therein, and being further provided with a first through-hole in communication with the detection chamber and a second through-hole in communication with the reference chamber thereon, such that a target gas is able to flow into the detection chamber and the reference chamber via the first through-hole and the second through-hole;
a light source, being disposed in the component housing, and being configured for emitting an incident light that enters the detection chamber and the reference chamber;
a colorimetric sensor film, being disposed in the detection chamber so as to be exposed to the target gas, and comprising a substrate and a sensing layer formed on the substrate; wherein the sensing layer is able to change a visual color thereof after adsorbing a specific chemical substance contained in the target gas through physisorption or chemisorption, such that after the incident light is directed to the sensing layer in the detection chamber, a reflective light response to the visual color is generated, thereby being adopted as a detection light;
a detection optical sensor, being disposed in the component housing so as to face the colorimetric sensor film, and being configured to generate a detected spectral signal after receiving the detection light;
a reference sensor film, being disposed in the reference chamber so as to be exposed to the target gas; wherein after the incident light is directed to the reference sensor film, a reflective light of the incident light is generated from a light incident surface of the reference sensor film, thereby being adopted as a reference light;
a reference optical sensor, being disposed in the component housing so as to face the reference sensor film, and being configured to generate a reference spectral signal after receiving the reference light; and
a processor, being coupled to the detection optical sensor and the reference optical sensor, and being configured to:
receive the detected spectral signal so as to correspondingly generate a detection data;
receive the reference spectral signal so as to correspondingly generate a reference data; and
generate a sensing data by comparing the detection data with the reference data;
wherein the sensing layer is made from a material that comprises at least one selected from a group consisting of carbon quantum dots, single-walled carbon nanotubes (SWCNTs), multi-walled carbon nanotubes (MWCNTs), graphene quantum dots (GQDs), M13 bacteriophage, short chain peptide, porphyrin, porphyrin derivative, metal-organic framework (MOF), phthalocyanine, phthalocyanine derivative, phthalein, and pH dye.

12. The modular sensor device of claim 11, wherein the substrate has a surface selected from a group consisting of smooth surface and rough surface, and the surface has a reflectivity of at least 80% in a wavelength range of 400-700 nm.

13. The modular sensor device of claim 11, wherein the substrate is selected from a group consisting of silicon substrate, glass substrate, porous substrate, polymer substrate, and silicon wafer.

14. The modular sensor device of claim 11, wherein a protective coating is formed on the substrate for cladding the sensing layer.

15. The modular sensor device of claim 11, wherein the sensing layer is enclosed in an encapsulation layer that is formed on the substrate.

16. The modular sensor device of claim 11, wherein a first recess across the first through-hole is formed on the component housing, and a second recess across the second through-hole is further formed on the component housing, such that the colorimetric sensor film and the reference sensor film are positioned in the first recess and the second recess, respectively.

17. The modular sensor device of claim 11, wherein a first optical path between the light source and the colorimetric sensor film has a first length, and a second optical path between the light source and the reference sensor film has a second length that is equal to the first length.

18. The modular sensor device of claim 16, further comprising a cap member, and the cap member comprising:
a third recess, being formed on a bottom of the cap member, and having a first bottom through-hole;
a fourth recess, being formed on the bottom of the cap member, and having a second bottom through-hole; and
a fifth recess, being formed on the bottom of the cap member, and having a third bottom through-hole;
wherein the cap member is disposed in the component housing for accommodating the detection optical sensor and the reference optical sensor and facing the colorimetric sensor film and the reference sensor film by the bottom thereof;
wherein the light source is disposed to face the third bottom through-hole, and the detection optical sensor and the reference optical sensor being disposed to face the first bottom through-hole and the second bottom through-hole, respectively.

19. The modular sensor device of claim 18, further comprising a circuit board connected to an opening of the cap member, wherein the detection optical sensor, the reference optical sensor, and the processor are disposed on the circuit board.

20. The modular sensor device of claim 18, further comprising a temperature sensor and a humidity sensor disposed in the detection chamber or the reference chamber, and the temperature sensor and the humidity sensor being coupled to the processor.

21. The modular sensor device of claim 18, further comprising:
a diffusion lens disposed in the fifth recess;
a first protective glass disposed in the third recess; and
a second protective glass disposed in the fourth recess.
